(19) United States
(12) Patent Application Publication
(10) Pub. No.: US 2014/0079589 A1
(43) Pub. Date: Mar. 20, 2014

(54) SELF-SEALING FILTER FOR STERILIZATION

(71) Applicant: 3M INNOVATIVE PROPERTIES COMPANY, St..Paul, MN (US)

(72) Inventors: Kevin D. Landgrebe, Woodbury, MN (US); Susan K. Reed, North Oaks, MN (US); Daniel R. McIntyre, ST. Paul, MN (US)

(73) Assignee: 3M INNOVATIVE PROPERTIES COMPANY, St. Paul (US)

(21) Appl. No.: 14/118,657

(22) PCT Filed: May 10, 2012
(86) PCT No.: PCT/US2012/037315

§ 371 Date: Nov. 19, 2013

Related U.S. Application Data
(60) Provisional application No. 61/490,344, filed on May 26, 2011.

(51) Int. Cl. A61L 2/26; A61L 2/16

(52) U.S. Cl. CPC A61L 2/26; A61L 2/16
USPC 422/28; 96/417; 96/4; 96/227; 206/525

(57) ABSTRACT

Filter assemblies that comprise a temporary attachment mechanism disposed on a surface of a filter are disclosed, as well as methods and systems related to using such filter assemblies during sterilization. The disclosed filter assemblies can include sterilization indicator elements.

### BACKGROUND

A variety of products and articles, including, for example, medical instruments, devices, and equipment, must be sterilized prior to use to prevent bio-contamination of a wound site, a sample, an organism, or the like. Sterilization of items used in medical procedures is vital to minimizing the spread of harmful and infectious agents to patients. Typically, the items used in medical procedures are placed into a container and wrapped using a sterilization wrap made of a gas permeable material or placed into a reusable vented rigid container. These sterilization containers are typically designed to preserve sterility of the items contained therein, as well as the interior portion of these containers, after the containers and contents of the container have been channeled through a sterilization procedure. During a typical sterilization procedure, the sterilization-wrapped containers or vented rigid containers are placed into a sterilization chamber, and the gas permeable material in the sterilization wrap or vents within the rigid container allows a sterilant to contact the item to be sterilized. Examples of such sterilants include steam, ethylene oxide, hydrogen peroxide, gas plasma, ozone, peracetic acid and the like.

A potentially useful class of sterilization containers features one or more openings or vents that are equipped with filters. The filters form a barrier to the unwanted penetration of microorganisms into the interior of the container, while still permitting the exchange of sterilant. Typically, the filter is placed in a support frame that is placed in and mechanically secured to the container opening. Such filters help ensure the instruments may be stored in the container until needed without increased risk of contamination after the initial sterilization. Furthermore, the filter may be discarded and the container reused after sufficient cleaning. The realities of hospital sterilization processing, however, render the use of these containers costly and inefficient.

One such problem stems from the trays used by manufacturers and vendors to group instruments. Related instruments are typically provided in metal trays that usually have a plurality of holes to facilitate sterilization. These trays may contain, for example, a set or sets of orthopedic implantable devices and tools. The vented sterilization containers described above are most often made of metal and can weigh up to 12 pounds, when empty. When they are loaded with a typical “vendor” tray for, say, an orthopedic implant procedure, the combined weight of the instrument trays plus the metal sterilization case could result in a filled container that weighs nearly 30 pounds. This is impractical and detrimental for workers to handle repeatedly throughout the work day. Many hospitals are now enforcing guidelines restricting the weight of sterilization packages.

As noted above, sterilization wraps provide a lighter weight alternative to the use of containers. Unlike rigid containers, however, sterilization wraps may be particularly susceptible to punctures or other failures during storage or transit.

Certain vented sterilization containers rely on disposable filters which need to be replaced before the container is again used to sterilize equipment. Different sterilization containers may rely on different mechanisms and structure for securing the filter support frame to the container opening and/or housing. Hospital personnel accordingly must be careful to replace the filters according to the mechanism or sealing structure of each different model of sterilization container they use. Maintaining and identifying a plurality of filter supports for a plurality of containers requires a substantial time investment and can result in inadequately sterilized instruments.

### SUMMARY

The present disclosure provides reusable or disposable filter assemblies that may be secured proximate an opening of a sterilization container. The disposable filter assembly includes a temporary attachment mechanism (e.g., an adhesive tape) secured to a filter material. In certain embodiments, the temporary attachment mechanism includes an indicator element that changes color to indicate exposure to sterilant. Filter assemblies may further be provided with carrier films to ease handling and to protect the attachment mechanism.

The present disclosure also provides for sterilization containers that include a filter assembly adhered proximate an opening in the container housing. The filter assembly may be secured to an interior surface of the container or an exterior surface of the container. In certain embodiments, the filter assembly includes sterilization indicator tape. Further, the sterilization container may include a plurality of openings to promote exchange of media (e.g., sterilants). A filter assembly is accordingly secured proximate each opening of the plurality of openings.

A sterilization container according to the present disclosure may comprise a lid and a tray that cooperate to form a housing. In some embodiments, a substantial portion of the interior volume of the housing is defined by the tray. In certain embodiments, a substantial portion of the interior volume is defined by the lid. The tray and/or the lid may also be formed of a substantially transparent material adapted to withstand exposure to steam, ozone, hydrogen peroxide, peracetic acid, and ethylene oxide without degradation of the lid and/or tray.

The present disclosure also provides for methods of sterilizing materials. The method includes providing a sterilization container having a tray, a lid, and opening for exchange of media. The material to be sterilized may be placed in the tray and the lid subsequently coupled to the tray. A filter assembly comprising a filter and a temporary attachment mechanism is affixed proximate the opening on a surface of the container. The method also includes inserting the sterilization container into a sterilization chamber for a length of time sufficient to sterilize the contents; and removing the sterilization container from said sterilization chamber. The method may further include verifying a change in a characteristic of an indicator element.

The filter assemblies of the present disclosure may be easily secured to a wide variety of sterilization containers without the need for intricate mechanical coupling. The use of a temporary attachment mechanism to align the filter and container opening provides for an effective barrier seal while decreasing the cost of disposing a filter. In embodiments wherein the attachment mechanism includes indicator elements, the exposure to sterilant may be easily verified without disturbing the interior of the chamber.

The present disclosure provides a container for sterilizing and storing material, the container comprising:
a housing;
an opening in the housing;
a filter covering at least a portion of the opening;
a temporary attachment mechanism disposed on at least a portion of the filter, the temporary attachment mechanism comprising an adhesive layer, wherein the adhesive layer is affixed to at least a portion of the filter and a portion of the housing.

The present disclosure further provides a container for sterilizing and storing material, the container comprising:
a housing;
an opening in the housing;
a filter covering at least a portion of the opening;
a temporary attachment mechanism disposed on at least a portion of the filter, wherein the temporary attachment mechanism is detachably affixed to at least a portion of the filter and a portion of the housing, wherein the temporary attachment mechanism is one of a reclosable fastener, a microstructured surface, and a magnetized substrate.

The present disclosure also provides for a disposable filter assembly for use in sterilizing material, the filter assembly comprising
a filter;
a sterilization indicator element; and
a temporary attachment mechanism affixed to a first surface of the filter, wherein the temporary attachment mechanism is disposed on and extends over at least a portion of the first surface,

The present disclosure further provides a method for sterilizing a material, the method comprising:
providing a housing including an interior and an opening;
placing the material in the housing;
covering the opening with a filter assembly, the filter assembly including a filter and a temporary attachment mechanism disposed on at least a portion of the filter, the temporary attachment mechanism including an adhesive layer;
securing a portion of the adhesive layer to the housing, thereby creating a barrier seal around the periphery of the opening; and
exposing the material to a sterilant.

The terms “indicator tape” and “sterilization indicator tape” mean an adhesive tape having chemical indicators that change color in response to sufficient exposure to sterilant.

As used herein, “continuous” means extending substantially across a surface and including no deliberate gaps or interruptions other than those inherent in the material.

The terms “comprises” and variations thereof do not have a limiting meaning where these terms appear in the description and claims.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As recited herein, all numbers should be considered modified by the term “about”.

As used herein, “a,” “an,” “the,” “at least one,” and “one or more” are used interchangeably. Thus, for example, a sterilization container including “an” opening can be interpreted to mean that the container includes “one or more” openings.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which can be used in various combinations. In each instance, the recited list serves only as a representative group and should not be interpreted as an exclusive list.

### BRIEF DESCRIPTION OF THE DRAWINGS

FIG. 1 is a perspective view of a sterilization container according to certain embodiments of the present disclosure.

FIG. 2 is a perspective view of a filter assembly according to certain embodiments of the present disclosure.

FIG. 3 is a cross-sectional view of a filter assembly according to certain embodiments of the present disclosure.

FIG. 4a is a perspective view of a filter assembly according to certain embodiments of the present disclosure.

FIG. 4b is a cross-sectional view of the filter assembly of FIG. 4a.

FIG. 5a is a cross-sectional view of a filter assembly secured to a container surface according to certain embodiments of the present disclosure.

FIG. 5b is a cross-sectional view of a filter assembly secured to a container surface according to certain embodiments of the present disclosure.

FIG. 5c is a cross-sectional view of a filter assembly secured to a container according to another embodiment of the present disclosure.

FIG. 6 is an exploded view of a sterilization container according to certain embodiments of the present disclosure.

FIG. 7 is a cross-sectional view of the sterilization container of FIG. 6.

FIG. 8 is a cross-sectional view of a sterilization container according to another embodiment of the present disclosure.

FIG. 9 is a cross-sectional view of a sterilization container according to certain embodiments of the present disclosure.

### DETAILED DESCRIPTION OF THE ILLUSTRATIVE EMBODIMENTS

The present disclosure provides for filter assemblies that may be secured proximate sterilization container openings. In certain embodiments, the filter assembly includes a filter and a temporary attachment mechanism. In some embodiments, the temporary attachment mechanism is disposed about the periphery of the filter to define at least a portion of the filter assembly boundary. The temporary attachment mechanism can be permeable or non-permeable to gas and/or vapor. The filter assembly can further include indicator elements.

The present disclosure also provides for sterilization containers having disposable filter assemblies secured thereto. The sterilization container includes a tray and a lid that cooperate to form a container housing. The container housing includes at least one opening to allow for exchange of sterilant. A filter assembly comprising a filter and temporary attachment mechanism is secured proximate the opening such that the filter covers at least a portion of the opening. In certain embodiments, the lid defines a substantial portion of the interior volume of the housing.

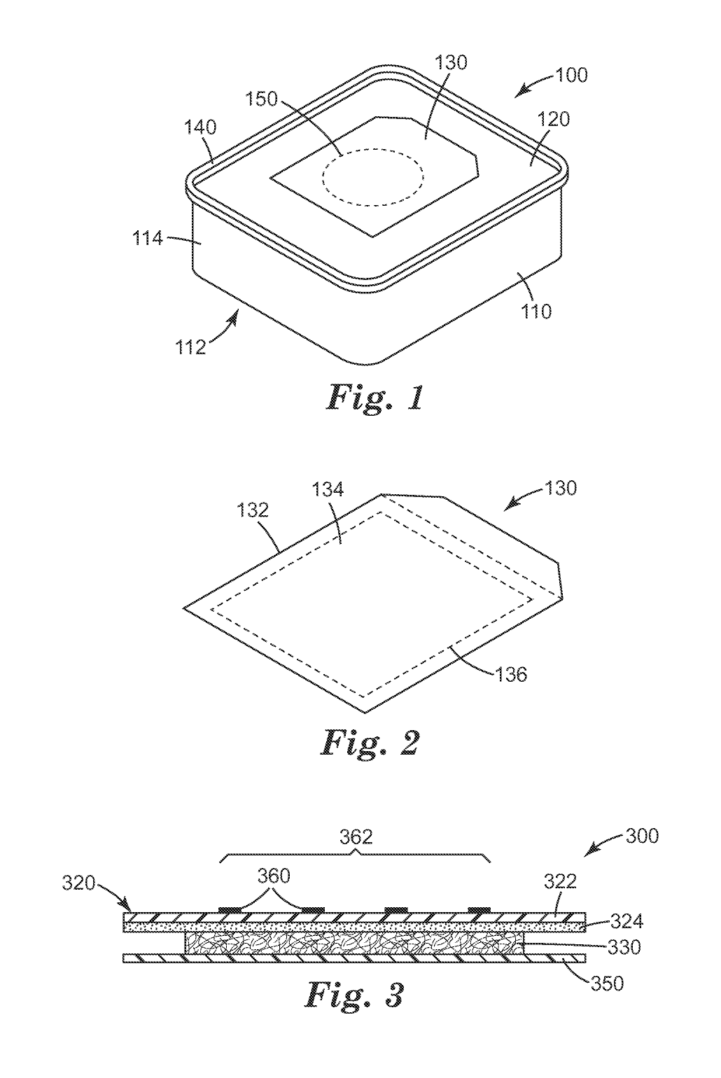

FIG. 1 depicts a filter assembly 130 of the present disclosure affixed to the lid 120 of a sterilization container 100. The sterilization container 100 includes a tray 110. The tray 110 includes a bottom wall surface 112 and a plurality of side walls 114. The lid 120 cooperates with the tray 110 to form the container housing.

The lid 120 or the tray 110 may include one or more locking mechanisms that allow the peripheral portion 140 of the lid 120 to fixedly engage the tray 110. Suitable locking mechanisms include, but are not limited to projections that extend from the peripheral portion 140 of the lid and that fixedly engage a rim of the tray 110. Alternative locking mechanisms include other known mating features distributed between the lid 120 and the tray 110. The tray and lid may also be coupled to one another using other known coupling means, including but not limited to an adhesive or adhesive tape.

The tray 110 and the lid 120 may be made of any material that allows the tray to withstand the temperature and level of sterilant exposure required for sterilization of the tray without degradation of the tray. That is, the tray 110 and lid 120 can be able to withstand temperatures of from about 100 degrees centigrade to 300 degrees centigrade without melting, bending, or losing strength. The tray 110 and/or lid 120 can be made of a transparent material that allows a viewer of the tray to visualize the contents of the tray by utilizing normal human visual acuity without opening the tray. Further, the tray 110 and/or the lid 120 can be made of a material that permits electromagnetic waves to be transmitted through the material so that the contents of the tray may be electronically interrogated. Alternatively, the lid 120 and/or tray 110 can include a transparent portion that acts as a window to the interior of the container.

Suitable materials for use in the tray 110 and/or lid 120 include, but are not limited to, various plastics including polyethylenes and polypropylenes, as well as metals well known in the art. Particularly suitable materials for high temperature sterilization conditions include but are not limited to plastics such as polyetherimide, polyphenylene oxide, polyphenylsulphone, polyethersulfone, and polycarbonates. In certain embodiments, the container housing comprises a plastic lid and a metal tray, thereby potentially reducing the weight of the container assembly. In other embodiments, the container can comprise a plastic lid and tray, a metal lid and tray, or a metal lid and plastic tray.

In the depicted embodiment, the lid 120 includes an opening 150 in communication with the filter assembly 130. The opening 150 allows for exchange of sterilant into the chamber when the lid 120 is coupled to the tray 110. It is contemplated, however, that any surface of the housing can include an opening. For example, the bottom wall 112 or a side wall 114 can include the opening. Furthermore, the housing can include a plurality of openings. In certain embodiments, a sterilization container includes an opening in both the lid 120 and the bottom wall 112 of the tray 110. In certain embodiments, one or more surfaces of the housing include a plurality of openings.

A filter assembly 130 is secured to a portion of the housing (e.g., the lid), proximate the opening 150 to ensure that the passage of pathogenic microorganisms into the housing after sterilization is sufficiently reduced or preferably prevented. Passage of pathogenic microorganisms can be reduced or prevented by creating a continuous seal around the periphery of the opening. In embodiments including a plurality of openings in distinct surfaces, a filter assembly is secured to a portion of the housing proximate each opening. Alternatively, a single filter assembly 130 may be secured to a surface of the housing such that the filter assembly covers a plurality of openings.

The filter assembly 130 of FIG. 1 is secured to an exterior surface of the housing. In other embodiments, the filter assembly 130 may be secured to an interior surface of the housing. In yet other embodiments, multiple filter assemblies are secured to the exterior of the housing, the interior of the housing, or combinations thereof. For example, one filter assembly may be secured to the exterior surface of a housing proximate to and covering an opening and another filter assembly may be secured to the interior of the housing proximate to and covering the same opening. As another example, a filter assembly may be secured proximate to and covering an opening on the lid and another filter assembly may be secured proximate to and covering another opening on the tray.

FIG. 2 depicts one embodiment of a filter assembly 130 including a filter 134 and a temporary attachment mechanism 132. In certain preferred embodiments, the temporary attachment mechanism 132 comprises an adhesive tape including a backing layer and an adhesive layer. Exemplary adhesive tapes include, but are not limited to Low Static Polyimide Film Tape 5419, available from 3M Company, St. Paul, Minn. In other embodiments, the temporary attachment mechanism 132 can be, for example, an adhesive disposed directly on a surface of the filter 134, a reclosable fastener, or a magnetized substrate adhered to the filter. The temporary attachment mechanism can include cooperating features (e.g., microreplicated features or other microstructures) on the surface of the housing proximate an opening.

The temporary attachment mechanism 132 is secured to at least a portion of the filter 134. In the embodiment depicted in FIG. 2, the temporary attachment mechanism 132 extends past the periphery 136 of the filter 134, such that the temporary attachment mechanism 132 defines at least a portion of the boundary of the filter assembly 130. In certain preferred embodiments, the temporary attachment mechanism 132 defines a substantial portion of the filter assembly boundary, such that a continuous or at least substantially continuous seal may be created between the attachment mechanism and a container proximate the filter periphery 136.

In the depicted embodiment, the temporary attachment mechanism 132 comprises a single continuous layer of one or more materials. In other embodiments, the attachment mechanism comprises a series of discrete segments. These segments may be disposed on the filter side by side or may be disposed in an overlapping manner. In other embodiments, the temporary attachment mechanism comprises segments disposed in a frame about the periphery of the filter, with a portion of filter remaining uncovered (i.e., having no attachment mechanism disposed thereon).

FIG. 3 depicts a cross-section of a filter assembly 300 according to one particular implementation. The filter assembly 300 includes a filter 330 and an indicator tape 320 disposed on a first surface of the filter 330. A carrier film 350 (if provided) is disposed opposite a second surface of the filter 330. An indicator element 360 is associated with (e.g., absorbed or coated onto) the backing layer 322, at least within an indicating region 362. The indicating region 362 comprises at least a portion of the backing layer 322, such that an exposure to sterilant may be verified by visual inspection.

The indicator tape 320 includes a backing layer 322 disposed on an adhesive layer 324. In the embodiment depicted in FIG. 3, the backing layer 322 is coextensive with the adhesive layer 324. In other embodiments, however, the backing layer 322 can extend beyond the adhesive layer 324 and vice versa. The filter assembly can also include a tab portion of the backing layer having no adhesive disposed thereon. The tab portion can be used, for example, to remove the filter assembly from a container after sterilization. Suitable indicator tapes include but are not limited to 3M 1222 and 1322 autoclave tapes and 3M Comply EO indicator tape, both available from 3M Company, St. Paul, Minn.

The indicator element 360 may be a compound or composition and is chosen such that the element undergoes a distinct and generally permanent color change upon exposure to conditions that may be used to verify the occurrence of sterilization (e.g., exposure to sterilant). In certain preferred implementations, the indicator element is embodied in an indicator ink. Steam sterilization indicator inks, such as lead carbonate-sulfur indicator inks and bismuth indicator inks, ethylene oxide sterilization indicator inks, or combinations thereof, can be associated with the backing layer 322 or the filter 330. Additional and exemplary indicator inks may be found, for example, in U.S. Pat. No. 5,916,816 (Read) and U.S. Pat. No. 5,780,098 (Battles), and Provisional Application No. 61/357,059 (Attorney Docket No. 66706US002), entitled CHEMICAL INDICATOR COMPOSITIONS, INDICATORS AND METHODS. Other indicators that may be associated with the backing include indicators for hydrogen peroxide, ozone, peracetic acid, any sterilant, and combinations thereof.

The indicator element 360 can include a binder material or resin. The binder holds the indicator compound or composition in place when coated on a substrate (e.g., backing layer). The binder can comprise a film-forming material, which is stable to heat and exposure to sterilant. A film formed by the binder is sufficiently permeable to sterilant to allow a desired color change to occur upon exposure to sterilant and/or other sterilization conditions. Materials that the binder may comprise include, for example, acrylate and methacrylate polymers and copolymers (e.g., poly(methylmethacrylate) and methyl/n-butyl methacrylate copolymer), poly(vinyl acetate) and poly(vinylchloride) and copolymers thereof, and various derivatives of cellulose, including, for example, ethylcellulose and nitrocellulose. In certain embodiments, the binder may be an ultraviolet light-, visible light-, or thermally-curable material.

Indicator elements can also optionally include other ingredients such as colorants that do not change color during a sterilization process, resins that perform functions other than binding (e.g., providing water resistance or solvent dispersibility), and opacifying agents. The indicator element can be applied to a backing or filter by any suitable method, including but not limited to coating, printing or absorbing.

The backing layer 322 can be made of any material that does not substantially degrade during sterilization. Suitable materials include, but are not limited to, nonwoven, woven, and knit webs, porous films (e.g., provided by perforations or microporous structure), foams, blotter paper, crepe paper, cardboard, plastic sheeting, metalized sheeting, metal foil and other known backings. In embodiments featuring indicator elements associated with a backing layer, the backing layer 322 can be made of any material which provides the necessary support for the indicator element but does not substantially interfere with its indicative function. The backing layer 322 can also be composed of more than one layer. For example, a microporous film can be laminated to a non-woven material to form a laminate backing layer material construction. These layers of film and material may or may not be coextensive. Additional materials suitable for use as backing may be found, for example, in U.S. Pat. No. 5,780,098 (Battles) and U.S. Pat. No. 6,534,006 (Hehenberger).

The backing layer 322 can provide additional strength and durability to the filter assembly 300, potentially reducing the incidence of puncture and failure of the filter before, during, and after sterilization. In addition to or in lieu of the backing layer 322, additional layers of material may be disposed between the filter 330 and the adhesive layer 324 and/or opposite the second surface of the filter 330. Suitable materials for use as the additional layer include nonwoven webs, including but not limited to Reemay® polyester filtration media, and Typar® polypropylene filtration media (both available from Fiberweb, Inc. Old Hickory, Tenn.), as well as any other materials suitable for use in a backing layer or the filter. Further suitable materials for use as an additional, strengthening layer include porous/perforated screens, lattices, and scrims comprising metals or polymers.

The adhesive layer 324 is disposed on at least a portion of a second surface of the backing layer 322 opposite the indicator composition and can be continuous, discontinuous, pattern coated, melt-blown, or combinations thereof. The adhesive layer 324 may include any number of adhesive materials. In certain embodiments, the adhesive layer 324 comprises a pressure sensitive adhesive (“PSA”). Examples of PSAs useful in the present disclosure include rubber-based adhesives (e.g., tackified natural rubbers, synthetic rubbers, and styrene block copolymers), (meth)acrylics (i.e., (meth)acrylates), poly(alpha-olefins), polyurethanes, and silicones.

Various PSAs can be used to form adhesive layer 324 on the backing layer 322 to make it adhesive. One well-known means of identifying PSAs is the Dahlquist criterion. This criterion defines a PSA as an adhesive having a 1 second creep compliance of greater than 1×10⁻⁶ cm²/dyne as described in Handbook of PSA Technology, Donatas Satas (Ed.), 2^{nd} Edition, p. 172, Van Nostrand Reinhold, New York, N.Y., 1989. Alternatively, since modulus is, to a first approximation, the inverse of creep compliance, PSAs may be defined as adhesives having a Young's modulus of less than 1×10⁶ dynes/cm². Another well known means of identifying a PSA is that it is aggressively and permanently tacky at room temperature and firmly adheres to a variety of dissimilar surfaces upon mere contact without the need of more than finger or hand pressure, and which may be removed from smooth surfaces without leaving a residue as described in Glossary of Terms Used in the Pressure Sensitive Tape Industry provided by the Pressure Sensitive Tape Council, 1996. Another suitable definition of a suitable PSA is that it preferably has a room temperature storage modulus within the area defined by the following points as plotted on a graph of modulus versus frequency at 25° C.: a range of moduli from approximately 2×10⁵ to 4×10⁵ dynes/cm² at a frequency of approximately 0.1 radians/sec (0.017 Hz), and a range of moduli from approximately 2×10⁶ to 8×10⁶ dynes/cm² at a frequency of approximately 100 radians/sec (17 Hz) (for example see FIG. 8-16 on p. 173 of Handbook of PSA Technology (Donatas Satas, Ed.), 2^{nd} Edition, Van Nostrand Rheinhold, New York, 1989). Any of these methods of identifying a PSA may be used to identify suitable PSAs for use in the methods of the present disclosure.

The material used to fabricate the filter 330 can be any material that is substantially permeable to fluid sterilants (e.g., steam, ethylene oxide, peroxide, and/or ozone), and that has filtration properties sufficient to prevent the passage of pathogenic microorganisms therethrough. In some embodiments, the filter material comprises a nonwoven material. The filter material can be fabricated from a variety of materials including, but not limited to, medical grade paper, rayon, polyolefin, polyester, polylactic acid, or a combination of any two or more of the foregoing materials. The filter material can be made using a variety of processes including, but not limited to, air laying processes, wet-laid processes, meltblowing processes, staple fiber carding and bonding processes, and solution spinning processes. In some embodiments, a filter can consist of a three-ply laminate of spunbond polypropylene/meltblown polypropylene/spunbond polypropylene (i.e., an “SMS” tri-laminate).

Filters can be generally characterized as falling into two main classes: reusables and disposables. Reusables are materials which, as the name suggests, can be reused, typically after washing or some other form of cleaning. Disposables, on the other hand, are usually single-use items that are discarded or recycled after their initial use. Generally, disposables include nonwovens (e.g., paper and fibrous polymeric materials) made from either or both natural and synthetic fibers, as well as films that are capable of passing sterilants and retarding transmission of bacteria and other contaminants.

Nonwoven filters have become particularly well-liked due to their barrier properties, economics and consistent quality. The nonwoven materials can be made from a variety of processes including, but not limited to, air laying processes, wet laid processes, hydroentangling processes, spunbonding, meltblowing, staple fiber carding and bonding, and solution spinning. The fibers themselves can be made from a variety of both natural and synthetic materials including, but not limited to, cellulose, rayon, polyesters, polyolefins, polyamides, and many other thermoplastic materials. The fibers may be relatively short, staple length fibers, typically less than 3 inches, or longer and substantially more continuous fibers such as are produced by spunbonding and meltblowing processes. Whatever materials are chosen, the resultant filter must be compatible with the particular sterilization technique being used and must also provide both strength and barrier properties to maintain the sterile nature of the contents until use.

Polyolefin-based fibers and their resultant nonwovens can be particularly well-suited for the production of filters. Polypropylene spunbonded nonwovens such as those available Kimberly-Clark Corporation (Dallas, Tex.) can be used to impart certain desirable characteristics to the filter.

A spunbonded/meltblown/spunbonded material is made from three separate layers that are laminated to one another. The method of making these layers is known and described in U.S. Pat. No. 4,041,203 to Brock et al. The material of Brock et al. is a three layer laminate of spunbonded/meltblown/spunbonded layers, which is also commonly referred to by the acronym “SMS”. The two outer layers of SMS are a spunbonded material made from extruded polyolefin fibers laid down in a random pattern and then bonded to one another. The inner layer is a meltblown layer also made from extruded polyolefin fibers generally of a smaller diameter and sometimes having a more discontinuous length than the fibers in the spunbonded layers. As a result, the meltblown layer provides increased barrier properties due to its fine fiber structure which permits the sterilizing agent to pass through the fabric while preventing passage of bacteria and other contaminants. Conversely, the two outer spunbonded layers provide a greater portion of the strength factor in the overall laminate.

The filter assembly 300 can optionally include a carrier film 350 to protect the adhesive layer 324 and filter 330 until the assembly 300 is ready for use. For example, the carrier film 350 can cover the surface of the filter that will be in communication with a container opening, as well as the portion of the adhesive layer 324 that is to be fixed to a container surface. The carrier film 350 typically remains attached to the filter assembly until a user is ready to apply the assembly 300 proximate a container opening. To further facilitate removal, the carrier film 350 can include a tab which extends past the periphery of the tape 320. The carrier film 350 may be a single piece or multiple piece release liner, and may be part of or laminated to a package (not shown) containing the assembly, or merely enclosed along with the assembly within the package. The carrier film 350 may keep the adhesive layer 324 clean during storage and shipping of the filter assembly 300.

Carrier films suitable for use with the disclosure can be made of kraft papers, polyethylene, polypropylene, polyester or composites of any of these materials. The films are preferably coated with release agents such as fluorochemicals or silicones. For example, U.S. Pat. No. 4,472,480 describes low surface energy perfluorochemical liners. The liners are papers, polyolefin films, or polyester films coated with silicone release materials. Examples of commercially available silicone coated release papers include POLY SLIK® silicone release papers available from Loparex, Inc. (Willowbrook, Ill.) and other silicone release papers available from Rexam Release, Inc. (Bedford Park, Ill.).

In another embodiment, the carrier film comprises a backing material or additional material as described above. In such embodiments, the filter is essentially sandwiched between layers of backing or other material. In order to secure such a filter assembly to the container housing, a second adhesive layer can be disposed on the surface of the carrier opposite the filter. Additionally or alternatively, the carrier film may be smaller than the first adhesive layer, leaving a portion of the first adhesive layer available to secure the filter assembly to a container.

The indicator tapes described herein may further optionally include a barrier layer covering a backing layer and indicator element. A low adhesion backsize layer can also be used to cover the barrier layer. Materials that may be used for the barrier layer include, for example, acrylic polymers, urea-formaldehyde compositions, styrene butadiene rubbers, cellulose acetate, cellulose acetate propionate, cellulose acetate butyrate, ethyl cellulose, non-drying coconut oil alkyd, and acrylic modified alkyd. Materials that can be used for the low adhesion backsize layer or release layer include, for example, acrylic, urethane, and silicone polymers.

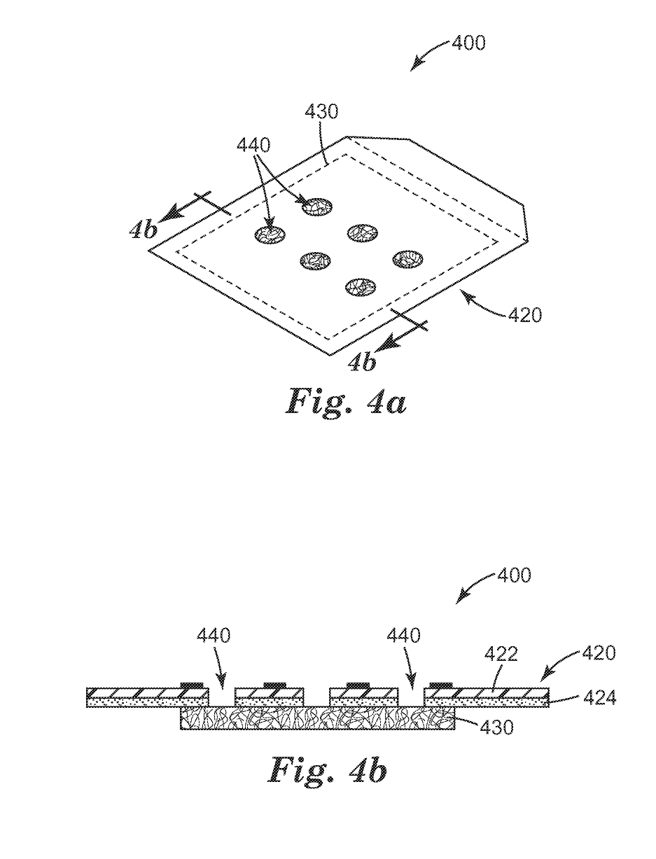

FIGS. 4a and 4b depict another embodiment of a filter assembly 400. With the exception of the indicator tape 420, the other elements of the filter assembly 400 are the same as those in FIG. 3 and need not be repeated. In certain embodiments, an indicator tape 420 may not be permeable to the sterilant due to lack of permeability of the backing layer 422 and/or the adhesive layer 424. To ensure adequate sterilization in such embodiments, the backing layer 422 and/or adhesive layer 424 can include apertures 440 formed therethrough to conduct sterilant through the filter 430 to the interior of the container and vice versa. The apertures may be provided as slits, voids or other openings sufficiently large enough to provide for the passage of sterilant through the indicator tape 420.

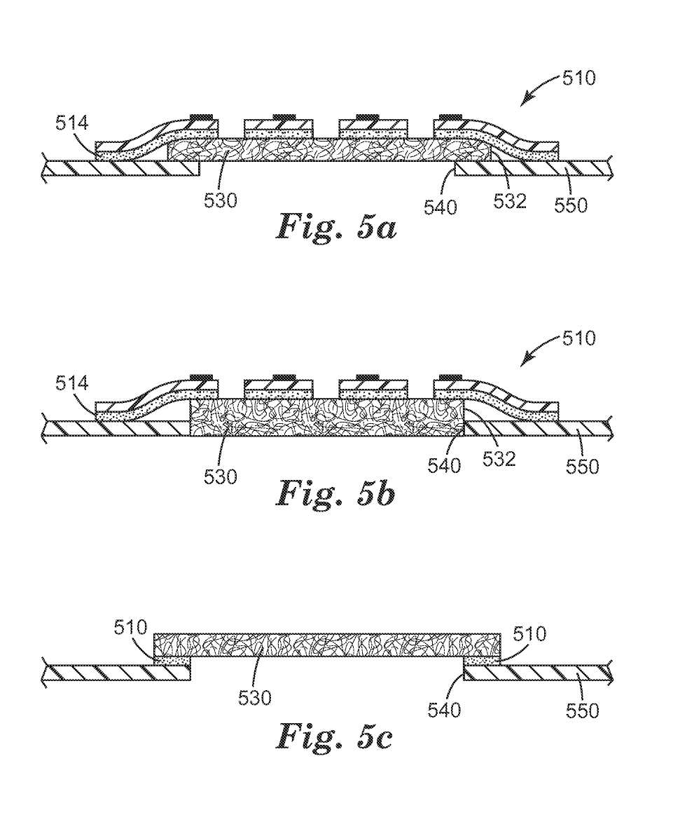

One exemplary attachment of a filter assembly to an opening is depicted in FIG. 5a. The attachment mechanism 510, in this embodiment an adhesive tape, extends a certain dimension past the edge 532 of the filter 530. The certain dimension is preferably of sufficient size for a barrier seal to be created between an adhesive layer 514 and the sterilization container housing surface 550. The attachment mechanism preferably creates a continuous barrier seal around the periphery of the opening 540, such that passage of pathogenic microorganisms into the housing after sterilization is sufficiently prevented.

The dimensions of the filter 530 can at least approximate the dimensions of the opening 540, such that filter 530 may sufficiently prevent contamination of the interior chamber. In the depicted embodiment, the filter 530 is the same size as, or larger than the opening 540. In certain embodiments and as depicted in FIG. 5b, the filter 530 is slightly smaller than and is received in the opening 540, and may be partially secured by a friction fit with a portion of the opening. Alternatively, a portion of the opening can be covered by other, typically impermeable, components of the filter assembly.

FIG. 5c depicts another exemplary attachment of filter 530 to a container surface 550. The temporary attachment mechanism 510 is disposed between the filter 530 and the container surface 550 proximate the opening 540. The temporary attachment mechanism 510 in such embodiments is preferably a pressure sensitive adhesive, a reclosable fastener, a magnetized substrate, or a series of microreplicated features (e.g., microstructures) that engage with the filter material.

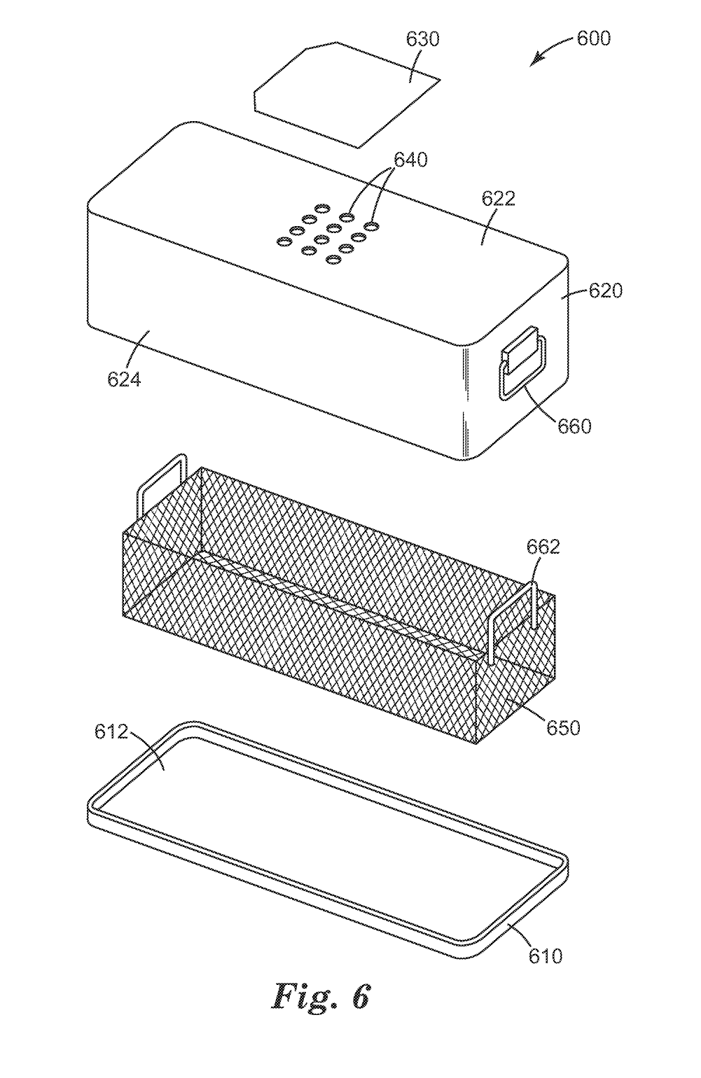

Referring to FIG. 6, a sterilization container 600 according to an embodiment of the present disclosure includes a container tray 610 and a container lid 620. The container lid 620 has a ceiling 622 and side walls 624 which define a portion of the interior volume. As can be appreciated, the lid 620 includes a plurality of openings 640 covered by a single filter assembly 630. A carrier (e.g., vendor tray) 650 is placed proximate the surface 612 of tray 610 for holding and transporting instruments and other items to be sterilized (not shown). The carrier 650 may be placed on the tray 610 or may be placed on projections (not shown) that support the carrier above the bottom surface. Both the carrier 650 and the lid 620 may include handles 660, 662 for easier manipulation.

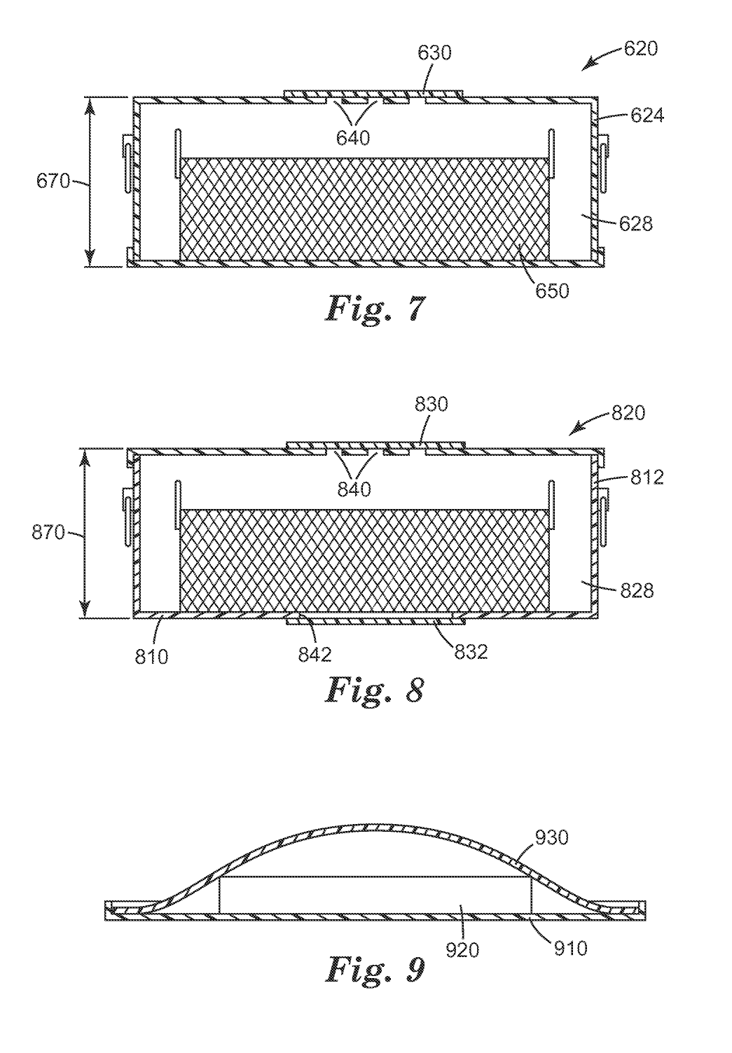

FIG. 7 depicts a cross-sectional view of the sterilization container of FIG. 6. The sterilization container comprises a height 670. In certain embodiments, the height of the lid side walls 624 defines at least 50% of the height 670. In other embodiments the height of the side walls 624 comprise at least 75% of the height, in other embodiments at least 90% and in yet other embodiments at least 95% of the height of the assembled container. As can be appreciated, the lid 620 defines a substantial portion of the interior volume 628. A sterilization container that includes a lid defining a substantial portion of the interior volume can be particularly advantageous. Once such a lid is removed, a user may lift the carrier from the tray without having to reach deep into the housing. This ergonomic improvement can increase efficiency and decrease the risk of accidental contamination.

Alternatively as shown in FIG. 8, the tray 810 defines a substantial portion of the interior volume 828, with the lid 820 acting as a covering. In certain embodiments, the height of the tray side wall 812 defines at least 50% of the container height 870. In other embodiments the height of the side wall 812 comprises at least 75% of the height 870, in other embodiments at least 90% and in yet other embodiments at least 95% of the height. In other embodiments not depicted herein, the lid and tray can be of equal or substantially equal height.

The sterilization container depicted in FIG. 8 also features openings 840, 842 and filter assemblies 830, 832 on opposite surfaces of the housing. The sterilization container interior may include additional indicator elements to indicate, for example, that the interior of the chamber has been exposed to sterilant and/or contains moisture.

The present disclosure also provides a method for sterilizing items. This method includes providing a sterilization container comprising a housing. The sterilization container includes a lid having two major surfaces and a peripheral portion. The peripheral portion of the lid is adapted to fixedly engage a tray. The sterilization container includes at least one opening therein. The sterilization container also includes a filter positioned in communication with the opening. The filter is gas and/or vapor permeable and is secured to the housing via a temporary attachment mechanism. The method also includes placing material inside the sterilization container; inserting the sterilization container into a sterilization chamber for a length of time sufficient to sterilize the materials; and removing the sterilization container from said sterilization chamber. The method can include securing the filter assembly to the container proximate the opening. Securing can include application of force to an adhesive, a magnet, a fastener, or use of additional methods of filter assembly securement known in the art. The method can further include the step of determining the effectiveness of the sterilization process by reference to an indicator element(s) on the exterior and/or interior of the housing. Reference can be characterized by visual inspection of color change or by electronic interrogation.

In yet another embodiment of the present disclosure depicted in FIG. 9, a filter assembly 930 is secured directly to a tray 910. The assembly exemplified by FIG. 9 may or may not include carrier 920 (i.e., material to be sterilized can be placed directly on the tray 910). In the depicted embodiment, the tray includes side walls that are shorter in height than the carrier 920. In other implementations, the tray includes side walls that are shorter in height than the materials to be sterilized. In yet other embodiments, the tray does not include side walls. The filter assembly 930 may be affixed to any portion of the tray not occupied by material. The filter assembly 930 is secured to the tray at least substantially surrounding the material to be sterilized. Preferably, at least a portion of the filter assembly 930 is gas and/or vapor permeable.

### EMBODIMENTS

1. A container for sterilizing and storing material, the container comprising: a rigid housing; an opening in a surface of the housing; a filter covering at least a portion of the opening; and a temporary attachment mechanism disposed on at least a portion of the filter, the temporary attachment mechanism comprising an adhesive layer, wherein the adhesive layer is affixed to at least a portion of the filter and a portion of the housing.
2. The container of embodiment 1, wherein the attachment mechanism comprises a backing layer.
3. The container of embodiment 2, wherein the temporary attachment mechanism includes at least one aperture through at least one of the backing and adhesive layers.
4. The container of embodiment 1, wherein the attachment mechanism is permeable to a sterilant.
5. The container of any of the preceding embodiments, wherein a portion of the temporary attachment mechanism extends beyond the periphery of the filter, and wherein the portion extending beyond the filter is affixed to the housing.
6. The container of any of the preceding embodiments, wherein at least one of the filter and the temporary attachment mechanism comprises a sterilization indicator element.
7. The container of any of the preceding embodiments, wherein the adhesive layer comprises a pressure sensitive adhesive.
8. The container of embodiment 1, wherein the temporary attachment mechanism comprises a plurality of segments.
9. The container of embodiment 8, wherein the segments are disposed on the filter in an overlapping arrangement.
10. The container of any of the preceding embodiments, wherein the filter comprises at least one of a nonwoven, a woven material, or paper.
11. The container of any of the preceding embodiments, wherein the housing comprises a tray including a base and a plurality of side walls.
12. The container of embodiment 11, wherein the housing further comprises a lid coupled to the tray.
13. The container of embodiment 12, wherein the lid comprises a plurality of side walls and a ceiling surface, and wherein the height of the lid side wall is greater than the height of the tray side wall.
14. The container of embodiment 12, wherein the lid comprises a plurality of side walls and a ceiling surface, and wherein the height of the tray side wall is greater than the height of the lid side wall.
15. The container of any of embodiments 11-14, wherein at least one of the tray and the lid comprises a transparent material.
16. The container of any of the preceding embodiments, wherein the housing comprises a metal tray coupled to a polymeric lid.
17. The container of embodiment 12, wherein the housing includes an interior volume, and wherein the lid defines a substantial portion of the interior volume of the housing.
18. The container of any of embodiments 11-17, wherein the housing comprises a plurality of openings.
19. The container of any of embodiments 11-17, wherein the lid and the tray each comprise at least one opening.
20. The container of any of the preceding embodiments, further comprising one or more additional layers of material between the filter and the adhesive layer and/or between the filter and the housing.
21. The container of any of the preceding embodiments, wherein affixation of adhesive layer to the housing forms a continuous barrier seal around a periphery of the opening.
22. The container of embodiment 1, wherein the housing comprises a plurality of openings in a surface thereof, and wherein the filter covers the plurality of openings.
23. A filter assembly for use in sterilizing material, the filter assembly comprising a filter; a sterilization indicator element; and a temporary attachment mechanism affixed to a first surface of the filter, wherein the temporary attachment mechanism is disposed on and extends over at least a portion of the first surface.
24. The filter assembly of embodiment 23, further comprising one or more additional layers of material between the filter and the temporary attachment mechanism.
25. The filter assembly of embodiment 23 or 24, wherein the temporary attachment mechanism comprises an adhesive layer and a backing layer.
26. The filter assembly of any of the preceding embodiments, wherein the temporary attachment mechanism is permeable to a sterilant.
27. The filter assembly of embodiments 23-25, wherein the temporary attachment mechanism comprises one or more apertures.
28. The filter assembly of any of the preceding embodiments, wherein at least a portion of the temporary attachment mechanism extends beyond the periphery of the filter.
29. The filter assembly of embodiment 28, wherein the filter assembly includes a frame and wherein the temporary attachment mechanism defines at least a portion of the frame.
30. The filter assembly of any of the preceding embodiments, wherein the temporary attachment mechanism comprises a pressure sensitive adhesive.
31. The filter assembly of any of the preceding embodiments, wherein the filter comprises at least one of a nonwoven, a woven material, or paper.
32. The filter assembly of any of the preceding embodiments, wherein the sterilization indicator element is capable of indicating exposure to steam, ozone, peracetic acid, hydrogen peroxide, ethylene oxide, or combinations thereof.
33. The filter assembly of any of the preceding embodiments, further comprising a carrier film attached to at least a portion of the temporary attachment mechanism.
34. The filter assembly of any of the preceding embodiments, wherein the filter comprises a second surface opposite the first surface, and wherein the filter assembly further comprises a layer of additional material proximate the second surface.
35. The filter assembly of any of the preceding embodiments and further comprising an item disposed on a surface of a tray, wherein a portion of the temporary attachment mechanism is coupled to at least a portion of the tray so that a barrier seal is created proximate the periphery of the item.
36. The filter assembly of embodiment 35, wherein the tray comprises a plurality of side walls, and wherein the temporary attachment mechanism is coupled to one or more of the side walls.
37. A method for sterilizing a material, the method comprising: providing a housing including a chamber and an opening; placing the material in the chamber; covering the opening with a filter assembly, the filter assembly including a filter and a temporary attachment mechanism disposed on at least a portion of the filter, the temporary attachment mechanism including an adhesive layer; securing a portion of the adhesive layer to the housing, thereby creating a barrier seal around the periphery of the opening; and exposing the material to a sterilant.
38. The method of embodiment 37, wherein the temporary attachment mechanism comprises an indicator element.
39. The method of embodiment 37 or 38, further comprising securing a lid to a tray to create the housing.
40. The method of any of the preceding embodiments, wherein the temporary attachment mechanism is permeable to sterilant.
41. The method of any of embodiments 37-39, wherein the temporary attachment mechanism comprises at least one aperture.
42. The method of any of the preceding embodiments, and further comprising verifying exposure to a sterilant.
43. The method of embodiment 42, wherein verifying the exposure to a sterilant comprises analyzing a sterilization indicator element on the exterior of the housing.
44. The method of embodiment 42 or 43, wherein verifying the exposure to a sterilant comprises analyzing a sterilization indicator element on the interior of the housing.
45. The method of any of the preceding embodiments and further comprising removing the filter assembly from the rigid housing.
46. The method of embodiment 45, wherein removing the filter assembly occurs after exposing the material to a sterilant.
47. The method of embodiment 45 or 46 and further comprising disposing of the filter assembly.
48. A container for sterilizing and storing material, the container comprising: a rigid housing; an opening in the housing; a filter covering at least a portion of the opening; a temporary attachment mechanism disposed on at least a portion of the filter, wherein the temporary attachment mechanism is coupled to at least a portion of the filter and a portion of the housing, wherein the temporary attachment mechanism comprises one of a reclosable fastener, a microstructured surface, and a magnetized substrate.

The complete disclosures of the patents, patent documents, and publications cited herein are incorporated by reference in their entirety as if each was individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1-30. (canceled)

31. A filter assembly for use in sterilizing material, the filter assembly comprising
a filter;
a sterilization indicator element; and
a temporary attachment mechanism affixed to a first surface of the filter, wherein the temporary attachment mechanism is disposed on and extends over at least a portion of the first surface;
wherein the temporary attachment mechanism comprises an adhesive layer and a backing layer.

32. The filter assembly of claim 31, wherein the temporary attachment mechanism is permeable to a sterilant.

33. The filter assembly of claim 31, wherein at least a portion of the temporary attachment mechanism extends beyond the periphery of the filter.

34. The filter assembly of claim 31, wherein the filter assembly includes a frame and wherein the temporary attachment mechanism defines at least a portion of the frame.

35. The filter assembly of claim 31, wherein the temporary attachment mechanism comprises a pressure sensitive adhesive.

36. The filter assembly of claim 31, wherein the filter comprises at least one of a nonwoven, a woven material, or paper.

37. The filter assembly of claim 31, wherein the sterilization indicator element is capable of indicating exposure to steam, ozone, peracetic acid, hydrogen peroxide, ethylene oxide, or combinations thereof.

38. A method for sterilizing a material, the method comprising:
providing a housing including a chamber and an opening;
placing the material in the chamber;
covering the opening with the filter assembly of claim 31;
securing a portion of the adhesive layer to the housing, thereby creating a barrier seal around the periphery of the opening; and
exposing the material to a sterilant.

39. The method of claim 38, wherein the temporary attachment mechanism comprises the indicator element.

40. The method of claim 38, wherein the temporary attachment mechanism is permeable to sterilant.

41. The method of claim 38, and further comprising verifying exposure to a sterilant.

42. The method of claim 38 and further comprising removing the filter assembly from the rigid housing after exposing the material to a sterilant.

43. A container for sterilizing and storing material, the container comprising:
a rigid housing;
an opening in a surface of the housing;
the filter assembly of claim 31;
wherein the filter covers at least a portion of the opening;
wherein the adhesive layer is affixed to at least a portion of the filter and a portion of the housing.

44. The container of claim 43, wherein a portion of the temporary attachment mechanism extends beyond the periphery of the filter, and wherein the portion extending beyond the filter is affixed to the housing.

45. The container of claim 43, wherein the temporary attachment mechanism comprises a plurality of segments.

46. The container of claim 43, wherein the housing comprises a tray including a base and a plurality of side walls.

47. The container of claim 46, wherein the housing further comprises a lid coupled to the tray.

48. The container of claim 43, wherein the housing comprises a plurality of openings.

49. The container of claim 48, wherein the housing comprises the plurality of openings in a surface thereof, and wherein the filter covers the plurality of openings.
